(19) United States
(12) Patent Application Publication
(10) Pub. No.: US 2025/0345145 A1
(43) Pub. Date: Nov. 13, 2025

(54) MEDICAL HOLDING ARM DEVICE

(71) Applicant: KARL STORZ SE & Co. KG, Tuttlingen (DE)

(72) Inventors: Sven Axel GRÜNER, Tuttlingen (DE); Jochen STEFAN, Tuttlingen (DE)

(21) Appl. No.: 18/868,342

(22) PCT Filed: May 25, 2023
(86) PCT No.: PCT/EP2023/064053

§ 371 Date: Nov. 22, 2024

(30) Foreign Application Priority Data
May 25, 2022 (DE) 10 2022 113 294.3

(51) Int. Cl. A61B 90/50; A61B 90/00; F16M 11/14

(52) U.S. Cl. CPC A61B 90/50; F16M 11/14; A61B 2090/064; A61B 2090/508; F16M 2200/066

(57) ABSTRACT

A medical holding arm device for a medical holding arm includes a holding arm segment, a further holding arm segment connected to the holding arm segment, a joint which connects the holding arm segment and the further holding arm segment to one another, a lock associated with the joint which locks the joint in a first operating state and releases it in a second operating state. The lock has a locking element for locking the joint and a transmission element for transmitting a force to the locking element. An actuator actuates the lock by exerting a force to transfer the locking mechanism from one operating state to the other. The medical holding arm device has a force sensor which at least indirectly detects a force exerted by the actuator on the lock in order to detect an operating state of the lock.

### CROSS REFERENCE TO RELATED APPLICATIONS

This application is a United States National Phase Application of International Application PCT/EP2023/064053, filed May 25, 2023, and claims the benefit of priority under 35 U.S.C. § 119 of German Application 10 2022 113 294.3, filed May 25, 2022, the entire contents of which are incorporated herein by reference.

### TECHNICAL FIELD

The invention relates to a medical holding arm device, a holding arm with such a holding arm device, a holding arm system with such a holding arm and an end effector.

### BACKGROUND

DE 10 2018 112 682 A1 proposes a medical holding arm device for a medical holding arm, with at least one holding arm segment, with a further holding arm segment connected at least indirectly to the holding arm segment, with a joint which connects the holding arm segment and the further holding arm segment to one another, with a lock associated with the joint which locks the joint in a first operating state and releases it in a second operating state and which comprises a locking element for locking the joint and a transmission element for transmitting a force to the locking element, and with an actuator which actuates the lock by exerting a force in order to transfer the lock from one operating state to the other.

The published patent application US 2020/0352676 A1 discloses a medical holding system with joints which can be motorically blocked and released.

The patent application DE 10 2021 114 151 A1 discloses a device for driving medical instruments with at least two motors.

The published patent application US 2019/0176334 A1 discloses a surgical robot system comprising a plurality of joints.

### SUMMARY

It is an object of the present invention in particular to provide a generic medical holding arm device with improved properties with regard to operational reliability. The object is achieved according to the invention by medical holding arm device and system features according to the invention. Advantageous embodiments and further developments of the invention are described.

The invention is based on a holding arm device for a medical, in particular surgical, holding arm, with at least one holding arm segment, with a further holding arm segment connected at least indirectly to the holding arm segment, with a joint which at least indirectly connects the holding arm segment and the further holding arm segment to one another, with a lock associated with the joint which locks the joint in a first operating state and releases it in a second operating state and which comprises a locking element for locking the joint and a transmission element for transmitting a force to the locking element, and with an actuator which actuates the lock by exerting a force in order to transfer the lock from one operating state to the other.

It is proposed that the holding arm device comprises a force sensor which detects an at least indirect force exerted by the actuator on the lock in order to detect an operating state of the lock.

This can advantageously improve operational safety. It can be particularly advantageous to check the operating state. A user can also be informed of the operating state during operation or even of incorrect operation.

The term “configured” should be understood to mean specifically programmed, provided, adapted, designed and/or equipped. The fact that an object is provided for a specific function should be understood in particular to mean that the object fulfills and/or executes this specific function in at least one application and/or operating state. A “holding arm device” is to be understood in particular as a functional component of a holding arm. The holding arm device can form the holding arm at least partially, preferably at least to a large extent. The term “at least to a large extent” should be understood to mean in particular at least more than 50%, preferably more than 70% and particularly preferably more than 90%, in particular with reference to a weight and/or a volume. Alternatively, the holding arm device can also form the holding arm completely. The holding arm is in particular configured for use during medical and in particular surgical interventions. For example, the holding arm can support a medical end effector. The end effector is, for example, an endoscope, exoscope or an endoscopic instrument, such as a clamp, forceps or the like. The end effector can be arranged at a distal holding arm segment of the holding arm device. The term “distal” should be understood in particular as facing a patient. The opposite of distal can be understood as proximal. The term “proximal” should be understood in particular as facing away from a patient. The holding arm device has at least two, preferably three and particularly preferably a plurality of holding arm segments which are at least indirectly connected to one another. The term “at least indirectly” should be understood to mean in particular indirectly but preferably directly. For example, a holding arm segment can be connected to another holding arm segment indirectly via an additional holding arm segment. Furthermore, two holding arm segments can be connected directly to one another. Two holding arm segments are advantageously connected to one another with a joint. The joint is in particular a ball joint, a hinge joint or the like. The joint is in particular formed at least partially, preferably at least to a large extent and particularly preferably completely by the two holding arm segments to be connected. The joint has at least one joint element which is connected to the holding segment or is at least partially formed integrally therewith. The term “at least partially connected and/or formed integrally” should be understood to mean in particular that at least one component of at least one object, in particular the object itself, is connected and/or formed integrally with at least one component of at least one further object, in particular with the further object itself. The term “integral” should be understood to mean in particular at least materially connected, for example by a welding process, an adhesive process, an injection molding process and/or another process that appears reasonable to a person skilled in the art. Advantageously, the term “integral” should also be understood to mean “one-piece”. The term “one-piece” should be understood in particular to mean in particular formed in one piece. Preferably, this one piece is produced from a single blank, a mass and/or a casting, particularly preferably in an injection molding process, in particular a single-component and/or multi-component injection molding process. Furthermore, the joint has in particular at least one further joint element which is connected to the further holding arm segment and is preferably at least partially formed integrally therewith. The further joint element is advantageously designed to correspond to the joint element. The joint element is designed in particular as a joint head, while the further joint element is designed as a counterpart to this as a joint socket. The fact that “a lock is associated with a joint” is to be understood in particular to mean that a respective lock is configured to determine the operating states of a respective associated joint. The lock is in particular a clamping mechanism in which the joint elements are clamped together so that they can no longer move freely relative to one another. It is therefore in particular a mechanical brake. Alternatively, other types of locking are also conceivable, such as a magnetic brake, a hydraulic brake or the like. The locking element exerts a force in particular on at least the joint element and/or the further joint element in order to lock them together and preferably to clamp them. The locking element can, for example, be designed as a cone tapering toward the distal side. In particular, the locking element is arranged within the holding arm segment and/or the joint. Preferably, the holding arm device has a locking element for each joint. The transmission element can in particular be formed at least partially integrally with the locking element. The transmission element is preferably arranged within the holding arm segment and/or the joint. The transmission element is designed in particular as a transmission line and advantageously as a transmission rod. Alternatively, the transmission element could be a flexible wire or the like. Furthermore, the transmission element could also be designed for hydraulic power transmission in the form of a fluid line. In particular, the number of joints is less than a number of holding arm segments, preferably one less. Preferably, the holding arm device has a transmission element and/or a locking element for each joint.

The force sensor can be arranged on the joint, in particular on the joint element and/or the further joint element of the joint. In order to further improve the sensitivity of the measurement by means of the force sensor, it is proposed that the force sensor is arranged on the lock, in particular on the transmission element of the lock. In particular, the force sensor is configured to measure the force by detecting an exerted pressure or strain.

It is further proposed that the holding arm device comprises an end segment, a further joint which at least indirectly connects the end segment and the further holding arm segment to one another, and a further lock associated with the further joint which blocks the further joint in a first operating state and releases it in a second operating state. Advantageously, a locking effect can be achieved up to an end segment of the holding arm device. An end segment can in particular be a distal and/or a proximal holding arm segment, i.e. in particular a last or a first holding arm segment.

It is proposed that the lock and the further lock are connected in series with one another, so that at least part of a force acting on the locking mechanism by the actuator can be transferred from the lock to the further lock, wherein these form a kinematic chain, so that the locks can always be switched together from one operating state to the other. Control can advantageously be simplified. In particular, the locking elements and the transmission elements are arranged one behind the other in series, so that the movement of a transmission element can be transferred to a locking element and from the locking element to a further transmission element and a further locking element.

In order to reduce complexity and/or reduce costs for sensors, it is also proposed that the force sensor is arranged on the further lock in order to detect an operating state of all locks. In particular, the entire holding arm device comprises only this one force sensor, which is arranged on the further lock and in particular the further transmission element of the further lock. In other words, preferably all further locks are free of force sensors.

In an advantageous embodiment of the invention, the force sensor can be designed as a strain gage. Component costs can be advantageously reduced. Alternatively, the force sensor could also be a piezoresistive pressure sensor; this is particularly useful if the locking element and/or the transmission element are pneumatic or hydraulic.

It is also proposed that the holding arm device comprises a control unit which is configured to read the force sensor in order to deduce an operating state of at least one lock. The monitoring of the operating states of the lock can advantageously be automated. The control unit comprises in particular at least one processor. Furthermore, the control unit may comprise a memory. In particular, an operating program is stored in the memory. The operating program is in particular executable by the processor. In order to read the force sensor, the control unit is connected to the force sensor electrically and/or electronically. For example, the control unit can be connected to the force sensor via cable. Alternatively, it is also conceivable that the control unit and the force sensor are connected wirelessly, for example via WLAN, RFID, Bluetooth or the like.

It is proposed that the control unit is configured to output an optical and/or acoustic signal when at least one lock is in the first operating state. Safety can advantageously be further improved because the signal can alert a user to the operating state of the lock. In particular, the optical and/or acoustic signal can be forwarded and/or output to an external device, such as a display, a loudspeaker, a screen, a handheld device, such as a smartphone, a tablet, a smartwatch or the like.

It is proposed that a force sensor is arranged on each lock and/or each transmission element in order to individually deduce the operating states of all locks. Safety can advantageously be particularly increased, as each operating state of each lock can thus be checked individually. The force sensors are in particular connected individually to the control unit for reading. The control unit can output an optical and/or acoustic signal depending on a respective force sensor and the operating state of a respective lock determined by this sensor.

A medical holding arm, in particular a surgical holding arm, is proposed with at least one holding arm device. This makes it possible to provide a medical holding arm with the advantages of the holding arm device. The holding arm can in particular comprise at least two, preferably at least three and particularly preferably a plurality of holding arm devices.

A holding arm system, in particular a robotic holding arm system, is proposed, with one or more holding arms and one or more end effectors arranged on the holding arm. This makes it possible to provide a medical holding arm system with the advantages of the holding arm device. The holding arm system can in particular comprise at least two, preferably at least three and particularly preferably a plurality of holding arms.

Further advantages are evident from the following description of the drawings. The drawings show an embodiment of the invention. The drawings, the description and the claims contain numerous features in combination. A person skilled in the art will also, expediently, consider the features individually and combine them into useful further combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a medical holding arm system with a plurality of holding arms in a perspective view;

FIG. 2 is a schematic representation of a holding arm device of one of the holding arms of the holding arm system in a sectional view;

FIG. 3 is a schematic representation of a lock of the holding arm device in a first operating state in a sectional view;

FIG. 4 is a schematic representation of a lock of the holding arm device in a second operating state in a sectional view;

FIG. 5 is a schematic flowchart of a method for operating a holding arm device; and

FIG. 6 is a schematic representation of a further embodiment of a holding arm device in a perspective view.

### DESCRIPTION OF PREFERRED EMBODIMENTS

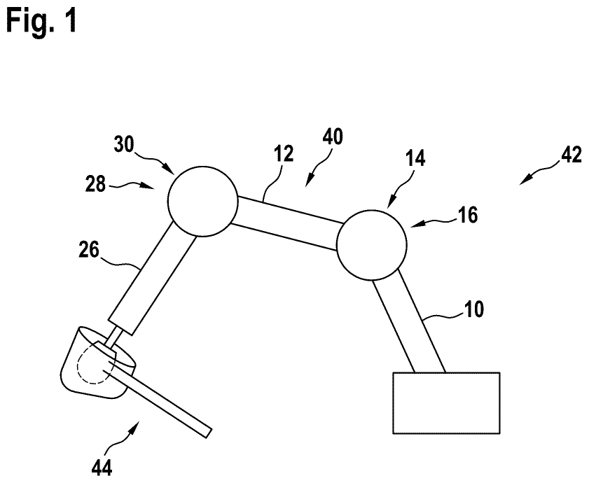

Referring to the drawings, FIG. 1 shows a schematic representation of an exemplary medical holding arm system 42. In the present case, the medical holding arm system 42 is designed as a manually operable medical holding arm system 42. Alternatively, it could also be a robotic medical holding arm system, which can be used for telesurgery, for example.

In the present case, the medical holding arm system 42 comprises, by way of example, a medical holding arm 40. Furthermore, the medical holding arm system 42 comprises an end effector 44. The end effector 44 is arranged on the holding arm 40. For this purpose, the end effector 44 is mounted to a distal end of the holding arm 8. The end effector 44 is configured to treat a patient. For this purpose, the end effector 44 may comprise an instrument, an endoscope or the like. It is conceivable that such a holding arm system can have a plurality of such medical holding arms and end effectors depending on the design and application.

In the present case, the medical holding arm 40 is configured for surgical use. The medical holding arm 40 comprises a medical holding arm device. In the present case, the holding arm device forms the holding arm 40 completely. Alternatively, the holding arm device can also represent only a part of the holding arm.

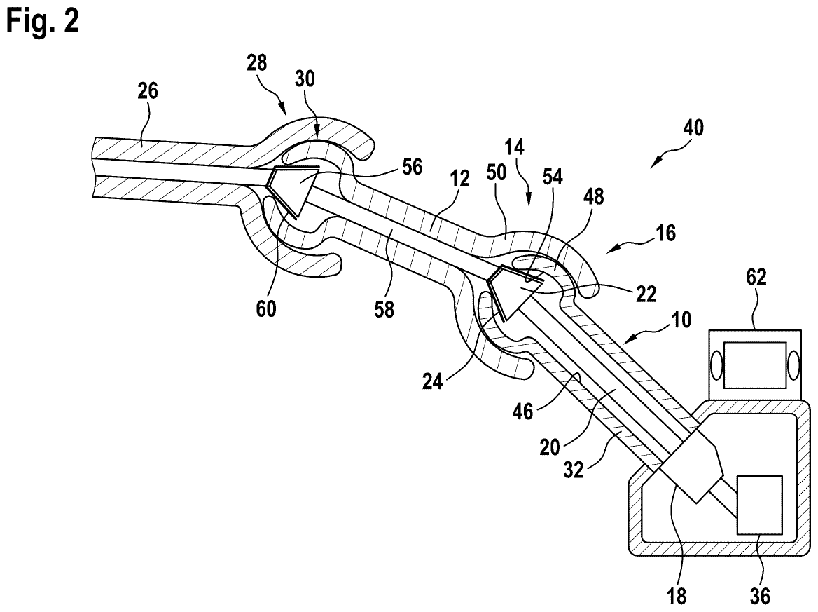

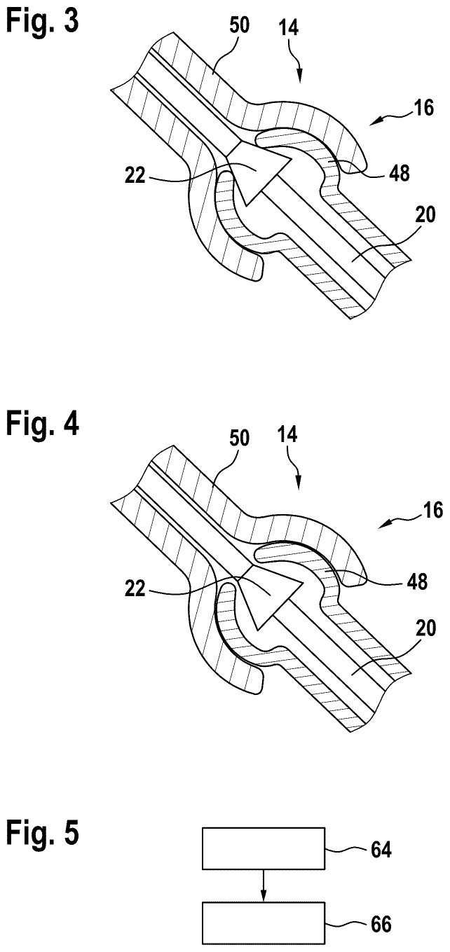

FIG. 2 shows a schematic representation of a part of the holding arm device in a sectional view. The holding arm device comprises at least one holding arm segment 10 (see FIG. 1). The holding segment 10 is arranged at the proximal end of the holding arm 40. The holding arm segment 10 comprises a transmission element 20. The transmission element 20 is configured for force transmission. In the present case, the transmission element 20 is configured for mechanical force transmission. The transmission element 20 is designed as a transmission rod. The transmission element 20 is rigid. Alternatively, a transmission element could be configured for pneumatic or hydraulic force transmission. For example, such a transmission element could be designed as a fluid line. Furthermore, it is conceivable that such a transmission element could be designed to be flexible.

The holding arm segment 10 has a support element 32. The support element 32 is configured to withstand a force exerted on the holding arm 40 from the outside, for example gravitational force, which is caused by the end effector 44 and/or the weight of the holding arm 40. Furthermore, the support element 32 serves to support other components, such as the transmission element 20 during force transmission along the holding arm 40.

In the present case, the support element 32 is designed as a tube. The carrier element 32 has a recess 46. The recess 46 extends along a longitudinal direction of the support element 32. The recess 46 is a longitudinal recess. The recess 46 is formed as a bore. The support element 32 receives the transmission element 20. The transmission element 20 is arranged in the recess 46 of the support element 32.

Furthermore, the holding arm device has a further holding arm segment 12. The further holding arm segment 12 is at least substantially identical to the holding arm segment 10. The further holding arm segment 12 and the holding arm segment 10 are at least indirectly connected to one another. In the present case, the further holding arm segment 12 and the holding arm segment 10 are even directly connected to one another. Accordingly, the further holding arm segment 12 also has a further transmission element 58.

The holding arm device comprises a joint 14. The joint 14 connects the holding arm segment 10 and the further holding arm segment 12 to one another. The joint 14 comprises a joint element 48. The joint element 48 is arranged on the support element 32 of the holding arm segment 10. The joint element 48 is arranged at the distal end of the support element 32. In the present case, the joint element 48 is connected integrally to the support element 32.

The joint 14 comprises a further joint element 50. The further joint element 50 is arranged on a further support element 52 of the further holding arm segment 12. The further joint element 50 is arranged at the proximal end of the further support element 52. In the present case, the further joint element 50 is connected integrally to the further support element 52.

The joint element 48 and the further joint element 50 engage with one another. The joint element 48 and the further joint element 50 are designed to correspond to one another. The joint element 48 is designed as a joint head. The further joint element 50 is designed as a joint head.

The holding arm device has at least one lock 16. The lock 16 is associated with the joint 14. The lock 16 has a first operating state. In the first operating state, the lock 16 locks the joint 14. Furthermore, the lock 16 has a second operating state. In the second operating state, the lock 16 releases the joint 14.

The lock 16 is arranged within the joint 14. The lock 16 has a locking element 22. The locking element 22 is arranged within the joint element 48. The locking element 22 is connected to the transmission element 20. The locking element 22 is trapezoidal in shape. The joint element 48 has a locking recess 54 corresponding to the locking element 22, in which the locking element 22 is arranged. In the first operating state of the lock 16, the locking element 22 is arranged with play in the locking recess 54. The locking element 22 can be displaced along the holding segment 10. In order to transfer the lock 16 to the first operating state, the locking element 22 can be displaced in the distal direction along the holding segment 10 into a locking position. In the locking position, the locking element 22 spreads the joint element 48, whereby this joint element is pressed against the further joint element 50. The joint elements 48, 50 are spread against one another and a frictional connection occurs between these two components. This first operating state is shown in FIG. 3 using a schematic representation of a part of the holding arm device in a sectional view. In order to transfer the lock 16 to the second operating state, the locking element 22 can be displaced in the proximal direction along the holding segment 10 into a release position. In the release position, the spreading of the joint element 48 is canceled, which in turn cancels the frictional connection between the joint element 48 and the further joint element 50.

Furthermore, the holding arm device comprises an additional holding arm segment 26 (see FIG. 1). In the present case, the additional holding arm segment 26 is an end segment which is located at the distal end of the medical holding arm 40. The end effector 44 is arranged on the additional holding arm segment 40. However, such an end effector can also be connected to such an additional holding arm segment via a joint, as described above.

In the present example, the additional holding arm segment 26 and the further holding arm segment 12 are directly connected to one another. However, it is conceivable that one or more holding arm segments and joints and associated locking devices are arranged between them.

Furthermore, the holding arm device has a further joint 28 for connecting the additional holding arm segment 26 and the further holding arm segment 12. The further joint 28 is at least substantially identical to the joint 14.

The holding arm device also has a further lock 30 for locking the further joint 28, which is associated with the further joint 28. The further lock 30 is at least substantially identical to the lock 16. A locking element 56 of the further lock 30 is connected to the further transmission element 58 of the further holding arm segment 12. The further locking element 56 is formed integrally with the further transmission element 58. The further transmission element 58 in turn rests on the locking element 22 of the lock 16. In this way, the transmission elements 20, 58 and the locking elements 22, 56 form a kinetic chain which can be moved uniformly. In other words, if a transmission element 20 of the holding arm segment 10 is moved, this movement is transferred to the locking element 22 and from there to the further transmission element 58 and in turn to the further locking element 56.

The holding arm device has at least one force sensor 24. The force sensor 24 detects a force exerted on the further lock 30 in order to deduce an operating state of at least the lock 16. In the present case, the holding arm device has a force sensor 24, 60 for each lock 16, 30. However, only a single force sensor can be used to determine the operating states of all locks, as long as the force sensor is associated with the last lock. In other words, this is possible if this single force sensor is at the end of the kinetic chain.

In the present case, the force sensor 24 is arranged, for example, on the locking element 22. More precisely, in the present case the force sensor 24 is arranged between the locking element 22 and the joint element 48. Alternatively, this could also be arranged on a further joint element or a further transmission element.

To actuate the locks 16, 30, the holding arm device has an actuator 16. The actuator 16 is arranged on the transmission element 20. The actuator 16 is designed, for example, as a linear motor.

For control purposes, the holding arm device has a control unit 36. The control unit 36 is configured to read the force sensors 24, 60 in order to deduce an operating state of at least one of the locks 16, 30. Furthermore, the control unit 36 controls the actuator 18. The control unit 36 comprises a memory (not shown). An operating program is stored in the memory. The operating program comprises a method for operating the holding arm device. The control unit 36 comprises a processor (not shown). The operating program is executable by means of the processor.

The control unit 36 is configured to output an optical and/or acoustic signal when one or all of the locks 16, 30 are in the first operating state. For this purpose, the holding arm device has an output apparatus 62. In the present case, the output apparatus 62 is designed as a screen with loudspeakers. By means of this output apparatus 62, both optical and acoustic signals can be output. For this purpose, the control unit 32 is electrically and/or electronically connected to the output apparatus 62.

FIG. 5 shows a schematic flowchart of an exemplary method for operating the holding arm device. The method is part of the operating program of the control unit 36.

The method comprises a method step 64. In the method step 64, the actuator 18 displaces the transmission element in the distal direction. Thus, the entire kinetic chain is shifted along the holding arm segments 10, 12 in the distal direction. This brings the locks 16, 30 into the first operating state. The joints 14, 28 are locked. The first operating state of the locks 16, 30 is detected by the force sensors 24, 60. To indicate that the first operating state has been reached, the control unit 36 outputs an optical and/or acoustic signal.

The holding arm device is thus protected against movement and can be used safely.

The method comprises a further method step 66. In the further method step 66, the actuator 18 displaces the transmission element 20 and thus in particular the entire kinetic chain along the holding arm segments 10, 12 in the proximal direction. The locks 16, 30 are brought into the second operating state. The joints 14, 28 are released. The second operating state of the lock will be detected by the force sensors 24, 60. To indicate that the second operating state has been reached, the control unit 36 outputs an optical and/or acoustic signal.

The holding arm device is thus made movable and can be transferred to any position, which can then be secured by returning to the first operating state according to the method step.

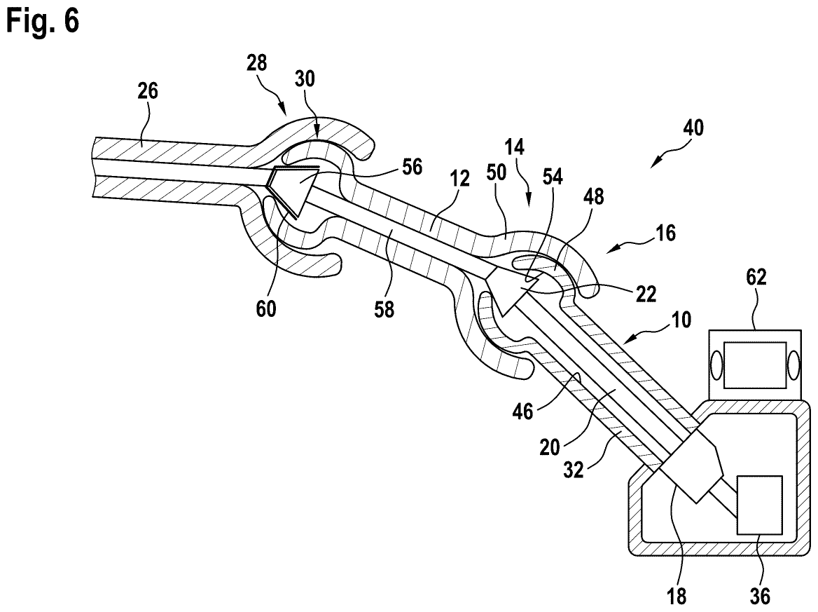

FIG. 6 shows a further embodiment of the holding arm device. The present embodiment differs from the previous one in that a single force sensor 60 is used to determine the operating states of all locks. The force sensor 60 is arranged between an additional holding arm segment 26, which is a distal end segment, and a further holding arm segment 12 of the holding arm device. Due to a force transmission across all locks 16, 30 and joints 14, 28, the operating states of all locks 16, 30 can be determined using a single force sensor 60.

A person skilled in the art will also, expediently, consider the features individually and combine them into useful further combinations.

The invention is based on a medical holding arm device for a medical, in particular surgical, holding arm 40, with at least one holding arm segment 10, with a further holding arm segment 12 connected at least indirectly to the holding arm segment 10, with a joint 14 which at least indirectly connects the holding arm segment 10 and the further holding arm segment 12 to one another, with a lock 16 associated with the joint 14 which locks the joint 14 in a first operating state and releases it in a second operating state and which comprises a locking element for locking the joint and a transmission element for transmitting a force to the locking element, and with an actuator 18 which actuates the lock 16 by exerting a force in order to transfer the lock from one operating state to the other. It is proposed that the medical holding arm device comprises a force sensor 24 which at least indirectly detects a force exerted by the actuator 18 on the lock 16 in order to detect an operating state of the locking mechanism 16.

While specific embodiments of the invention have been shown and described in detail to illustrate the application of the principles of the invention, it will be understood that the invention may be embodied otherwise without departing from such principles.

### LIST OF REFERENCE SIGNS

10 Holding arm segment
12 Further holding arm segment
14 Joint
16 Lock
18 Actuator
20 Transmission element
22 Locking element
24 Force sensor
26 Additional holding arm segment
28 Further joint
30 Further lock
32 Support element
36 Control unit
40 Medical holding arm
42 Medical holding arm system
44 End effector
46 Recess
48 Joint element
50 Further joint element
52 Further support element
54 Locking recess
56 Further locking element
58 Further transmission element
60 Force sensor
62 Output apparatus
64 Method step
66 Further method step

1. A medical holding arm device for a medical, in particular surgical, holding arm, the device comprising:
at least one holding arm segment;
a further holding arm segment connected at least indirectly to the holding arm segment;
a joint which at least indirectly connects the holding arm segment and the further holding arm segment to one another;
a lock associated with the joint which locks the joint in a first operating state and releases it in a second operating state and which comprises a locking element for locking the joint and a transmission element for transmitting a force to the locking element;
an actuator which actuates the lock by exerting a force in order to transfer the locking mechanism from one operating state to the other; and
a force sensor which at least indirectly detects a force exerted by the actuator on the lock in order to detect an operating state of the lock.

2. The medical holding arm device according to claim 1, wherein the force sensor is arranged on the lock.

3. The medical holding arm device according to claim 1, further comprising:
an additional holding arm segment configured as an end segment;
a further joint which at least indirectly connects the additional holding arm segment and the further holding arm segment to one another; and
a further lock associated with the further joint, which blocks the further joint in a first operating state and releases the further joint in a second operating state.

4. The medical holding arm device according to claim 3, wherein the lock and the further lock are connected in series with one another, so that at least part of a force exerted by the actuator on the lock can be transferred from the lock to the further lock, whereby the lock and the further lock form a kinetic chain, so that the locks can be switched together from one operating state to the other.

5. The medical holding arm device according to claim 3, wherein the force sensor is arranged on the further lock to detect an operating state of all locks.

6. The medical holding arm device according to claim 1, wherein the force sensor is configured as a strain gage.

7. The medical holding arm device according to claim 1, further comprising a control unit which is configured to read the force sensor to deduce at least one operating state of at least one lock.

8. The medical holding arm device according to claim 7, wherein the control unit is configured to output an optical and/or acoustic signal when at least one lock is in the first operating state.

9. The medical holding arm device at least according to claim 3, further comprising another force sensor wherein a respective one of the force sensors is arranged on each lock in order to individually deduce the operating states of all locks.

10. A medical holding arm, with a holding arm device comprising:
at least one holding arm segment;
a further holding arm segment connected at least indirectly to the holding arm segment;
a joint which at least indirectly connects the holding arm segment and the further holding arm segment to one another;
a lock associated with the joint which locks the joint in a first operating state and releases it in a second operating state and which comprises a locking element for locking the joint and a transmission element for transmitting a force to the locking element;
an actuator which actuates the lock by exerting a force in order to transfer the locking mechanism from one operating state to the other; and
a force sensor which at least indirectly detects a force exerted by the actuator on the lock in order to detect an operating state of the lock.

11. A medical holding arm system comprising:
one or more medical holding arms each of the one or more the medical holding arms comprising:
at least one holding arm segment;
a further holding arm segment connected at least indirectly to the holding arm segment;
a joint which at least indirectly connects the holding arm segment and the further holding arm segment to one another;
a lock associated with the joint which locks the joint in a first operating state and releases it in a second operating state and which comprises a locking element for locking the joint and a transmission element for transmitting a force to the locking element;
an actuator which actuates the lock by exerting a force in order to transfer the locking mechanism from one operating state to the other; and
a force sensor which at least indirectly detects a force exerted by the actuator on the lock in order to detect an operating state of the lock; and
one or more end effectors arranged on each of the one or more the medical holding arms.

12. The medical holding arm device according to claim 1, wherein the force sensor is arranged on the locking element of the lock.
